(19) United States
(12) Patent Application Publication
(10) Pub. No.: US 2007/0287964 A1
(43) Pub. Date: Dec. 13, 2007

(54) HYPODERMIC NEEDLE TIP PROTECTOR

(76) Inventors: Hooman Asbaghi, San Diego, CA (US); Don Millerd, San Diego, CA (US)

Correspondence Address: NYDEGGER & ASSOCIATES, 348 OLIVE STREET, SAN DIEGO, CA, 92103, US

(21) Appl. No.: 11/422,851

(22) Filed: Jun. 7, 2006

(51) Int. Cl. A61M 5/00; A61M 5/32; A61M 5/31

(52) U.S. Cl. 604/192; 604/187; 604/240

(57) ABSTRACT

A device is provided to protect the hypodermic needlepoint of a syringe from being dulled as it penetrates through the stopple of a fluid vial for the purpose of filling the syringe with fluid from the vial. Specifically, the device includes a blunt needle that covers the hypodermic needle and that interacts with the syringe to establish relative friction forces between them. Importantly, this friction force is less than the friction force subsequently established between the dull needle and the stopple. Thus, after the hypodermic syringe has been filled, and is withdrawn from the fluid vial, the stronger friction force between the stopple and the dull needle causes the dull needle to remain with the stopple as the hypodermic syringe is removed from the fluid vial.

### FIELD OF THE INVENTION

The present invention pertains generally to hypodermic syringes. More particularly, the present invention pertains to methods and devices for protecting the distal tip of a hypodermic needle from being dulled when it is used to penetrate the stopple of a fluid vial for the purpose of filling the syringe with fluid from the vial. The present invention is particularly, but not exclusively, useful as a method and device for employing static friction forces that will hold a protective cover (i.e. blunt needle) over the distal end of a hypodermic needle until after the hypodermic syringe has been filled with fluid from a fluid vial.

### BACKGROUND OF THE INVENTION

As is well known, fluid transfer systems or devices can be employed in many different procedures and applications, and for many different reasons. Perhaps one of the most well known applications requiring the transfer of a fluid from one container to another involves the filling of a hypodermic syringe with a fluid medicament. Although pre-filled syringes and disposable pre-filled fluid cartridges are often used, they do not satisfy all needs. There are still many applications wherein it is necessary, or desirable, to fill an empty or partially filled syringe with a fluid that is taken directly from a fluid vial.

Heretofore, the filling of a hypodermic syringe (or other similar device) with fluid medicament from a fluid vial has been done in a rather straightforward manner. Specifically, to perform such a task, the needle of the hypodermic syringe is inserted through the stopple of a medical fluid vial. The plunger of the syringe is then pulled back to transfer a desired amount of fluid from the vial into the fluid chamber of the syringe. Once this has been done, the hypodermic needle is then withdrawn from the stopple of the fluid vial and the hypodermic syringe is ready to be used.

Although the general procedure set forth above is simple and straightforward, there are still some drawbacks. For instance, it happens that during the insertion of a hypodermic needle into the stopple of a fluid vial, the needlepoint of the hypodermic needle can become significantly dulled. This, obviously, has an adverse effect on any subsequent use of the syringe for injecting fluid medicament into a patient.

In light of the above, it is an object of the present invention to provide a device for protecting the needlepoint of a needle of a hypodermic syringe from being dulled when it is used during a procedure to fill the syringe with fluid medicament from a fluid vial. Another object of the present invention is to provide a protective device for the needlepoint of a needle of a hypodermic syringe that is easily and automatically removed from the needle without requiring any additional manipulation, during a syringe filling procedure. Yet another object of the present invention is to provide a device for protecting the needlepoint of a hypodermic needle that is easy to use, is simple to manufacture, and is cost effective.

### SUMMARY OF THE INVENTION

In accordance with the present invention, a device is provided for protecting the needlepoint of a hypodermic needle as it (i.e. the needlepoint) is inserted through the stopple of a fluid medicament vial. Specifically, the device of the present invention includes a blunt needle that fits over the needlepoint at the distal end of the hypodermic needle. Importantly, the blunt needle is held on the hypodermic needle by only static friction forces until it (i.e. the blunt needle) is no longer needed.

For one embodiment of the present invention, the device includes a shrink wrap that is used to hold the blunt needle on the hypodermic needle. For this embodiment, the hypodermic needle is formed with an abutment that is located at a proximal distance “d” from the needlepoint. Importantly, relative to the hypodermic needle, the blunt needle has a length “l” that is greater than the distance “d” (l>d). Further, the blunt needle is formed with a lumen that is dimensioned for receiving the distal needlepoint of the hypodermic needle. Thus, the blunt needle can be advanced over the hypodermic needle until its proximal end urges against the abutment on the hypodermic needle. In this combination, the needlepoint of the blunt needle extends distally beyond the distal needlepoint of the hypodermic needle by approximately one eighth of an inch.

As indicated above, this first embodiment of the present invention also includes a shrink wrap. Specifically, part of the shrink wrap is positioned over the abutment and a portion of the hypodermic needle. The rest of the shrink wrap is then used to extend over the proximal end of the blunt needle. The purpose here is to have the shrink wrap hold the blunt needle on the hypodermic needle until it is to be removed, as described below. Importantly, with this combination, a static friction force “f₂” is established between the shrink wrap and the blunt needle. Also, a static friction force “f₃” is established between the shrink wrap and the hypodermic needle. An important aspect of the present invention is that “f₃” is a predetermined static friction force, and is much less than the static friction force “f₂” (f₃<<f₂).

For an alternate embodiment of the present invention, the hypodermic syringe body is formed with an annular recess that surrounds the proximal end of the hypodermic needle. This recess will be characterized by having an inner diameter (i.d.) while, on the other hand, the blunt needle has an outer diameter (o.d.). For this embodiment, the o.d. of the blunt needle and the i.d. of the recess are relatively dimensioned to establish an interference fit between them when the proximal end of the blunt needle is received into the recess of the syringe. As intended for the present invention this interference fit establishes a predetermined static friction force between the blunt needle and the syringe.

For the operation of the present invention, it is envisioned that the hypodermic syringe is to be filled with fluid from a fluid vial. To do this, because the fluid vial will typically include a stopple, the stopple needs to be penetrated by a sharp object, such as the needlepoint of the syringe. For the present invention, this penetration is to be accomplished by the blunt needle, while it is in position over the hypodermic needle. The purpose here is two-fold. For one, with this penetration, fluid communication is established between the vial and the syringe for filling the fluid chamber of said syringe. For another, a static friction force “f₁” is established between the blunt needle and the stopple. As required for the present invention, this static friction force “f₁” needs to be much greater than the predetermined static friction force established between the blunt needle and the syringe. Consequently, after the syringe has been filled, and after the hypodermic needle and syringe have been withdrawn from the fluid vial, the blunt needle will remain held by the stopple. The syringe can then be operationally used.

In operation, the blunt needle, while it is in place over the hypodermic needle, is inserted through the stopple of a fluid medicament vial. Importantly, this penetration of the stopple establishes a static friction “f₁” between the blunt needle and the stopple. For the shrink wrap embodiment of the present invention the static friction force “f₁” is comparable to the static friction force “f₂” between the shrink wrap and the blunt needle. On the other hand, a predetermined static friction force “f₃” is established between the shrink wrap and the hypodermic needle that is much less than either “f₁” or “f₂” (f₁≅f₂>>f₃). On the other hand, for the alternate embodiment of the present invention (i.e. no shrink wrap), it is only important that the static friction force “f₁” be greater than the predetermined static friction force that is generated by the interference fit between the blunt needle and the syringe body. In all embodiments, both the hypodermic needle and the blunt needle are preferably made of a stainless steel, the stopple is made of an elastomeric material, and the shrink wrap is made of a polymer well known in the pertinent art.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is an exploded perspective view of a device in accordance with the present invention;

FIG. 2 is a perspective view of the combination of a blunt needle engaged with a hypodermic needle in accordance with an embodiment of the present invention;

FIG. 3 is a cross-section view of the combination as seen along the line 3-3 in FIG. 2;

FIG. 4 is a cross-section view of the combination of a blunt needle engaged with a hypodermic syringe as seen along the line 4-4 in FIG. 1;

FIG. 5 is a perspective view of a hypodermic syringe engaged with a fluid vial during a filling of the hypodermic syringe; and

FIG. 6 is an elevation cross-sectional view of a fluid vial with a penetrating blunt needle after the blunt needle has been used for filling a hypodermic syringe.

### DESCRIPTION OF THE PREFERRED EMBODIMENTS

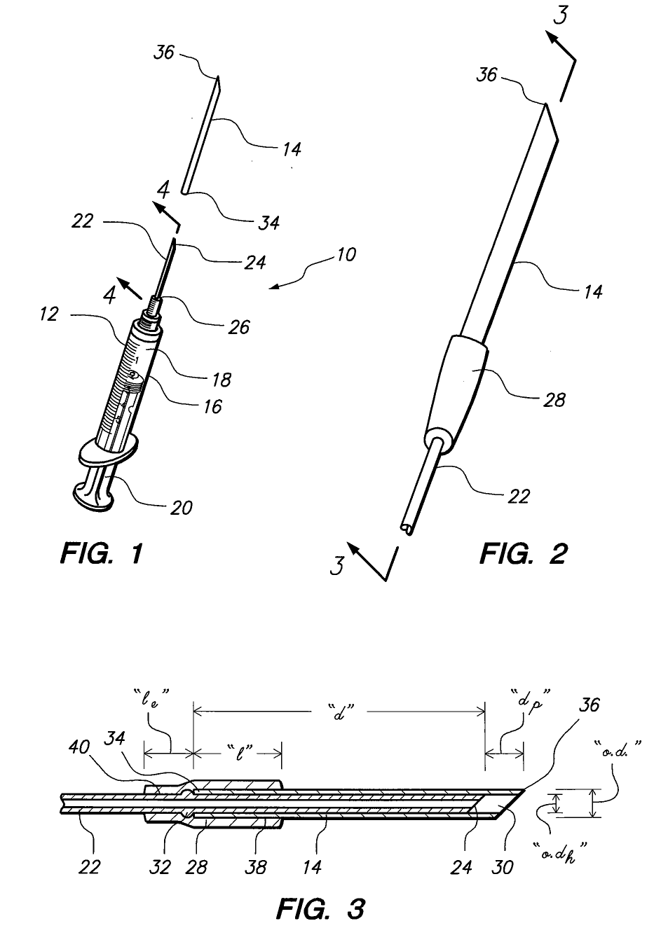

A device for filling a hypodermic syringe with a fluid medicament is shown in FIG. 1 and is generally designated 10. In FIG. 1 it will be seen that the device 10 includes a hypodermic syringe 12 and a blunt needle 14. For purposes of the present invention, the hypodermic syringe 12 that is shown is only exemplary. Accordingly, as will be appreciated by the skilled artisan, any device that is functionally similar to the hypodermic syringe 12 can be used. In general, such a device 10 will include a syringe body 16 that is formed with a fluid chamber 18. Also, there will be a plunger 20 that is mounted on the syringe body 16 for advancement into the fluid chamber 18 to expel fluid from the chamber 18 through a hypodermic needle 22. As shown, the hypodermic needle 22 will typically have a distal needlepoint 24 and a proximal end 26 that is mounted on the syringe body 16 for fluid communication with the fluid chamber 18.

Referring now to FIG. 2, the salient aspects of a preferred embodiment for the present invention are shown. In FIG. 2 it will be seen that this embodiment for the present invention includes a shrink wrap 28 that interconnects with both the blunt needle 14 and the hypodermic needle 22. In further detail, and as perhaps best seen in FIG. 3, the blunt needle 14 is formed with a lumen 30. Also, the hypodermic needle 22 is formed with an abutment 32 that is located at a distance “d” proximal from the distal needlepoint 24 of the hypodermic needle 22. As intended for the present invention, with this structure the hypodermic needle 22 is advanced into the lumen 30 of the blunt needle 14 until its abutment 32 comes in contact with the proximal end 34 of the blunt needle 14. The distal needlepoint 24 of the hypodermic needle 22 will then be located inside the lumen 30 of the blunt needle 14. It will also be positioned at a distance “dₚ” proximal to the distal needlepoint 36 of the blunt needle 14. For purposes of the present invention, the distance “dₚ” will preferably be about one eighth of an inch. The shrink wrap 28 is then used to hold the blunt needle 14 on the hypodermic needle 22.

Still referring to FIG. 3, it will be seen that approximately two thirds (⅔) of the shrink wrap 28 covers a length “l” along the portion of the blunt needle 14 at its proximal end 34. The remaining one third (⅓) of the shrink wrap 28 covers the abutment 32, as well as a portion of the hypodermic needle 22 along a length “lₑ” (i.e. l≅2lₑ). Further, it is shown that the outer diameter of the blunt needle 14 (o.d.) is greater than the outer diameter of the hypodermic needle 22 (o.d.ₕ). The consequence of these dimensional distinctions is manifested in the resultant static friction forces that are developed between the various components. Specifically, due to the greater area of contact between the shrink wrap 28 and the blunt needle 14, a static friction force “f₂” is established at their interface 38 that is much greater than the static friction force “f₃” that is established at the interface 40 between the shrink wrap 28 and the hypodermic needle 22. In addition to holding the blunt needle 14 on the hypodermic needle 22, the present invention envisions that the shrink wrap 28 will also create a fluid seal that will prevent fluid from leaking between the hypodermic needle 22 and the blunt needle 14.

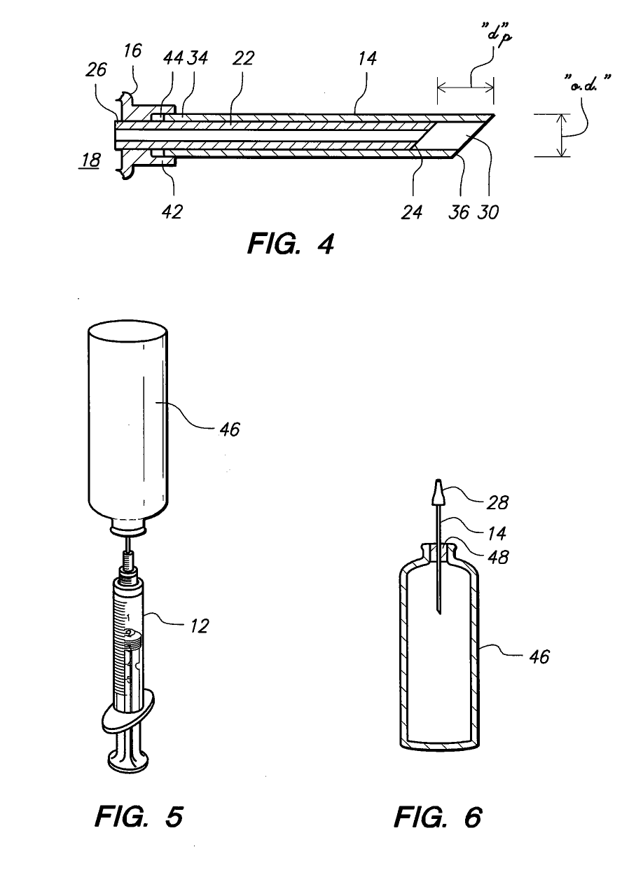

Referring now to FIG. 4, an alternate preferred embodiment for the device 10 of the present invention is shown that relies on a direct contact between the blunt needle 14 and the syringe body 16. Specifically, for this embodiment, the syringe body 16 includes a circular wall 42 that creates an annular recess 44 on the syringe body 16 that is characterized by an inner diameter (i.d.). With this structure, the proximal end 26 of hypodermic needle 22 is affixed to the syringe body 16 to extend distally through the annular recess 44, and beyond. Similar to the preferred embodiment disclosed above, for the alternate preferred embodiment, the hypodermic needle 22 is also inserted into the lumen 30 of the blunt needle 14. This time, however, the proximal end 34 of the blunt needle 14 becomes inserted into the annular recess 44 that is formed on the syringe body 16. With this in mind, the inner diameter (i.d.) of the annular recess 44 is dimensioned relative to the o.d. of the blunt needle 14 to allow for this insertion. Moreover, the selected dimensions also establish an interference fit between the recess 44 and the needle 14 that is characterized by a static friction force “f₄”. Also, and again similar to the other preferred embodiment disclosed above, when engaged with each other, the distal needlepoint 24 is positioned inside the lumen 30 of the blunt needle 14, and it is located at a proximal distance “dₚ” from the distal needlepoint 36 of the blunt needle 14.

As envisioned for the present invention, both the blunt needle 14 and the hypodermic needle 22 can be made of a stainless steel. Alternatively, the blunt needle 14 may be made of a plastic material, if desired. Also, it is envisioned for the present invention that the shrink wrap 28 can be made of any type material well known in the pertinent art that has a shrink ratio of approximately 2:1.

In operation, the hypodermic syringe 12 is provided, with the blunt needle 14 that is attached thereto as disclosed above. The blunt needle 14 is then inserted into a fluid vial 46, as shown in FIG. 5, for the purpose of filling the fluid chamber 18 of the syringe 12 with a fluid (e.g. a fluid medicament). For this task, it is necessary that the blunt needle 14 penetrate through a stopple 48 on the fluid vial 46 (see FIG. 6). The necessary force to do this is transferred from the syringe body 16 to the proximal end 34 of the blunt needle 14 as it urges against the abutment 32 (preferred embodiment), or against the syringe body 16 itself (alternate preferred embodiment). Importantly, once the blunt needle 14 has penetrated the stopple 48, a static friction force “f₁” is established between the blunt needle 14 and the stopple 48.

It is an important operational aspect of the present invention that all of the static friction forces established during the manufacture of the device 10 have a predetermined relationship to each other. Specifically, for the preferred embodiment (i.e. wherein the shrink wrap 28 is employed) it is important that the static friction force “f₁” established between the blunt needle 14 and the stopple 48 be much greater than the static friction force “f₃” that is established between the shrink wrap 28 and the hypodermic needle 22 (f₁>>f₃). As disclosed above for this embodiment, the static friction force “f₂” that is established between the shrink wrap 28 and the blunt needle 14 is also much greater than the friction force “f₃”. Further, in their general relationship to each other, “f₁” is preferably about equal to “f₂” and thus: f₁≅f₂>>f₃. For the alternate preferred embodiment of the device 10, it is important that the static friction force “f₁” established between the blunt needle 14 and the stopple 48 be much greater than the static friction force “f₄” that is established between the proximal end 34 of the blunt needle 14 and the annular recess 44 of the syringe body 16 (f₁>>f₄).

With the above in mind, after the blunt needle 14 has penetrated the stopple 48 and the hypodermic syringe 12 has been filled with fluid from the fluid vial 46, the hypodermic syringe 12 is withdrawn from the vial 46. Due to the significant differences in the respective static friction forces that are involved, the blunt needle 14 will remain stuck in the stopple 48 under the influence of the relatively large static friction force “f₁” (see FIG. 6). On the other hand, because “f₃” is the smallest friction force involved in the preferred embodiment (i.e. with shrink wrap 28) the hypodermic needle 22 of the hypodermic syringe 12 will be removed from the blunt needle 14. In this case the shrink wrap 28 will remain with the blunt needle 14 (see FIG. 6). Recall, f₂>>f₃. Further, the shrink wrap 28 that remains with the blunt needle 14 may help prevent subsequent fluid leakage from the vial 46. For the alternate preferred embodiment, because the “f₄” is less than “f₁”, the hypodermic syringe 12 will also be removed from the blunt needle 14.

For both embodiments of the present invention, the friction force “f₃” (preferred embodiment) and the friction force “f₄” (alternate preferred embodiment) are specifically designed and engineered. In detail, they are designed and engineered to establish predetermined static friction forces between the hypodermic syringe 12 and the blunt needle 14 that will be overcome by any static friction force that may result from the penetration of the dull needle 14 into the stopple 48.

While the particular Hypodermic Needle Tip Protector, as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A protective device which comprises:
a hypodermic syringe formed with a fluid chamber and having a hypodermic needle with a distal needlepoint and a proximal end, with the fluid chamber being in fluid communication with the hypodermic needle; and
a blunt needle having a distal needlepoint and a proximal end, said blunt needle being formed with a lumen for receiving the distal needlepoint of said hypodermic needle therein, wherein the proximal end of said blunt needle is engaged with said hypodermic syringe to establish a predetermined static friction force therebetween for holding said blunt needle on said syringe to cover and protect the needlepoint of the hypodermic needle until the predetermined static friction force is overcome by a force applied on said blunt needle in a distal direction.

2. A device as recited in claim 1 wherein the hypodermic needle is formed with an abutment at a proximal distance “d” from the needlepoint and the hypodermic needle is advanced into the lumen of the blunt needle until the proximal end of the blunt needle urges against the abutment on the hypodermic needle, and wherein the device further comprises a shrink wrap positioned over a portion of the hypodermic needle and over the proximal end of the blunt needle to establish the predetermined static friction force for holding said blunt needle on the hypodermic needle.

3. A device as recited in claim 2 wherein the syringe is to be filled with fluid from a vial, and the vial includes a stopple for penetration thereof by said blunt needle to establish fluid communication between the vial and the syringe for filling the fluid chamber of said syringe.

4. A device as recited in claim 3 wherein a static friction force “f₁” is established between the blunt needle and the stopple, wherein a static friction force “f₂” is established between the shrink wrap and the blunt needle, and wherein the predetermined static friction force is a static friction force “f₃” established between the shrink wrap and the hypodermic needle, and further wherein f₁≅f₂>>f₃.

5. A device as recited in claim 4 wherein the blunt needle has a length “l” and wherein “l” is greater than “d” by more than one eighth of an inch.

6. A device as recited in claim 1 wherein the blunt needle has an outer diameter (o.d.) and the syringe is formed with an annular recess surrounding the proximal end of the hypodermic needle, with the recess having an inner diameter (i.d.), and wherein the o.d. of the blunt needle and the i.d. of the recess are dimensioned to establish an interference fit with the predetermined static friction force therebetween when the proximal end of the blunt needle is received in the recess of the syringe.

7. A device as recited in claim 6 wherein the syringe is to be filled with fluid from a vial, and the vial includes a stopple for penetration thereof by said blunt needle to establish fluid communication between the vial and the syringe for filling the fluid chamber of said syringe, and wherein a static friction force “f₁” is established between the blunt needle and the stopple, with f₁ being much greater than the predetermined static friction force established between the blunt needle and the syringe.

8. A device as recited in claim 1 wherein the hypodermic needle and said blunt needle are made of a stainless steel.

9. An apparatus for filling a hypodermic syringe with a fluid medicament which comprises:
a vial;
a stopple for holding the fluid medicament in said vial;
a syringe body;
a hypodermic needle having a proximal end and a distal end with a needlepoint at its distal end and with its proximal end affixed to said syringe body for fluid communication therewith;
a blunt needle having a distal needlepoint and a proximal end, said blunt needle being formed with a lumen for receiving the distal needlepoint of the hypodermic needle therein; and
a means for engaging with the proximal end of said blunt needle to establish a predetermined static friction force for holding said blunt needle on the hypodermic needle to cover and protect the needlepoint of the hypodermic needle until the predetermined static friction force is overcome by a force applied on said blunt needle in a distal direction.

10. An apparatus as recited in claim 9 wherein the predetermined static friction force is established between said blunt needle and said syringe body.

11. An apparatus as recited in claim 9 further comprising a shrink wrap positioned over the hypodermic needle and over the proximal end of the blunt needle to hold said blunt needle on the hypodermic needle, and wherein the predetermined static friction force is established between the shrink wrap and the hypodermic needle.

12. An apparatus as recited in claim 9 wherein the hypodermic needle is formed with an abutment at a proximal distance “d” from the needlepoint and the hypodermic needle is advanced into the lumen of the blunt needle until the proximal end of the blunt needle urges against the abutment on the hypodermic needle, and wherein the apparatus further comprises a shrink wrap positioned over a portion of the hypodermic needle and over the proximal end of the blunt needle to establish the predetermined static friction force for holding said blunt needle on the hypodermic needle.

13. An apparatus as recited in claim 12 wherein the syringe body is to be filled with fluid from a vial, and the vial includes a stopple for penetration thereof by said blunt needle to establish fluid communication between the vial and the syringe for filling the fluid chamber of said syringe, wherein a static friction force “f₁” is established between the blunt needle and the stopple, wherein a static friction force “f₂” is established between the shrink wrap and the blunt needle, and wherein the predetermined static friction force is “f₃” and is established between the shrink wrap and the hypodermic needle, and further wherein f₁≅f₂>>f₃.

14. An apparatus as recited in claim 9 wherein the blunt needle has an outer diameter (o.d.) and the syringe body is formed with an annular recess surrounding the proximal end of the hypodermic needle, with the recess having an inner diameter (i.d.), and wherein the o.d. of the blunt needle and the i.d. of the recess are dimensioned to establish an interference fit with the predetermined static friction force therebetween when the proximal end of the blunt needle is received in the recess of the syringe, and further wherein the syringe is to be filled with fluid from a vial, and the vial includes a stopple for penetration thereof by said blunt needle to establish fluid communication between the vial and the syringe for filling the fluid chamber of said syringe, and wherein a static friction force “f₁” is established between the blunt needle and the stopple, with f₁ being much greater than the predetermined static friction force established between the blunt needle and the syringe.

15. A method for manufacturing an apparatus for filling a hypodermic syringe with a fluid medicament which comprises the steps of:
providing a hypodermic syringe having a hypodermic needle affixed thereto with its needlepoint extending therefrom in a distal direction;
placing a blunt needle over the hypodermic needle to cover and protect the needle point of the hypodermic needle during the filling of the hypodermic syringe; and
establishing a predetermined static friction force to hold the blunt needle over the hypodermic needle until the predetermined static friction force is overcome by a force applied on the blunt needle in a distal direction.

16. A method as recited in claim 15 wherein the establishing step comprises the steps of:
peening the hypodermic needle at a proximal distance “d” from its distal needlepoint to form an abutment thereon;
advancing the hypodermic needle into the blunt needle until the proximal end of the blunt needle urges against the abutment on the hypodermic needle; and
positioning a shrink wrap over a portion of the hypodermic needle and over the proximal end of the blunt needle to establish the predetermined static friction force.

17. A method as recited in claim 16 wherein the hypodermic syringe is to be filled with fluid from a vial, and the vial includes a stopple for penetration thereof by the blunt needle to establish fluid communication between the vial and the syringe for filling the fluid chamber of said syringe, wherein a static friction force “f₁” is established between the blunt needle and the stopple, wherein a static friction force “f₂” is established between the shrink wrap and the blunt needle, and wherein the predetermined static friction force “f₃” is established between the shrink wrap and the hypodermic needle, and further wherein f₁≅f₂>>f₃.

18. A method as recited in claim 15 further comprising the steps of:
dimensioning the blunt needle with an outer diameter (o.d.);
forming the syringe body with an annular recess surrounding the proximal end of the hypodermic needle, with the recess having an inner diameter (i.d.), and wherein the o.d. of the blunt needle and the i.d. of the recess are dimensioned to establish an interference fit with the predetermined static friction force therebetween when the proximal end of the blunt needle is received in the recess of the syringe.

19. A method as recited in claim 18 wherein the hypodermic syringe is to be filled with fluid from a vial, and the method further comprises the step of selecting a stopple for the vial for penetration thereof by said blunt needle, wherein a static friction force “f₁” is established between the blunt needle and the stopple, with f₁ being much greater than the predetermined static friction force established between the blunt needle and the syringe.

20. A method as recited in claim 19 wherein the blunt needle remains in the stopple to expose the needlepoint of the hypodermic needle upon withdrawal of the syringe from the vial.
